(19) United States
(12) Patent Application Publication
(10) Pub. No.: US 2025/0123290 A1
(43) Pub. Date: Apr. 17, 2025

(54) BIOTIN-FREE PROXIMITY LABELING

(71) Applicant: HOWARD HUGHES MEDICAL INSTITUTE, Chevy Chase, MD (US)

(72) Inventors: Shu-Hsien Sheu, Ashburn, VA (US); Pratik Kumar, Ashburn, VA (US); Luke D. Lavis, Ashburn, VA (US)

(21) Appl. No.: 18/915,759

(22) Filed: Oct. 15, 2024

Related U.S. Application Data
(60) Provisional application No. 63/590,534, filed on Oct. 16, 2023.

(51) Int. Cl. G01N 33/68; C07C 275/24; C07C 323/22; C07K 19/00; C12N 9/08

(52) U.S. Cl. CPC G01N 33/6848; C07C 275/24; C07C 323/22; C07K 19/00; C12N 9/0065; C07C 2601/16; C12Y 111/01011

(57) ABSTRACT

Compounds useful for biotin-free proximity labeling are provided. Also provided are methods for biotin-free labeling of biomolecules in proximity to a target in a cell, which involve attaching an engineered labeling enzyme to the target; and contacting the cell to a compound as disclosed, which is a substrate of the engineered labeling enzyme and which includes a label moiety, wherein the biomolecules in proximity to the target are labeled.

### RELATED APPLICATIONS

This application claims priority from U.S. Provisional Application Ser. No. 63/590,534 filed Oct. 16, 2023, the entire disclosure of which is incorporated herein by this reference.

### TECHNICAL FIELD

The presently-disclosed subject matter generally relates to compounds and methods for labeling biomolecules in proximity to a target in a cell. In particular, certain embodiments of the presently-disclosed subject matter relate to compounds and methods for biotin-free proximity labeling.

### INTRODUCTION

Proximity labeling refers to techniques used to identify and study interactions between biomolecules within a cellular context. Generally, the technique involves labeling biomolecules proximal to a protein of interest, and has traditionally made use of biotin for marking the proximal biomolecules for pulldown and analysis.

Proximity labeling has become a powerful tool for studying structures and interactions within cells. However, most techniques rely on biotinylation of proteins, and there are a number of limitations associated with the use of biotin in proximity labeling. For example, cells naturally contain biotin, which can contribute to false positives.

Another limitation of biotin-based proximity labeling is the potential for non-specific labeling. Biotinylation enzymes, such as biotin ligase, can sometimes label proteins that are not in close proximity to the protein of interest, leading to background noise and complicating data interpretation. This non-specific labeling can obscure true interactions and reduce the overall accuracy of the technique.

The efficiency of biotinylation can also vary depending on the cellular environment and the accessibility of the target proteins. In some cases, the biotinylation reaction may not proceed efficiently, resulting in incomplete labeling of proximal proteins. This can lead to underrepresentation of certain interactions and affect the comprehensiveness of the analysis.

The use of biotin also introduces challenges in downstream processing and analysis. Biotinylated proteins require specific conditions for effective pulldown and isolation, which can be technically demanding and time-consuming. Additionally, the presence of endogenous biotin in cells can interfere with the pulldown process, necessitating additional steps to reduce background noise and improve specificity.

The reliance on biotin can also limit the applicability of proximity labeling in certain experimental contexts. For example, in studies involving live animals or specific tissues, the poor cell permeability of biotin-phenol and its inability to cross the blood-brain barrier (BBB) restricts its use. This limitation necessitates the development of alternative labeling strategies that can overcome these barriers and enable more versatile applications.

Accordingly, there remains a need in the art for tools that can be used to perform biotin-free proximity labeling.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compounds and methods for biotin-free proximity labeling.

The presently disclosed subject matter includes a compound having the following structure of formula (I):

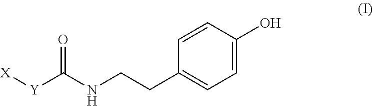

wherein X is a self-labeling protein ligand, and Y is

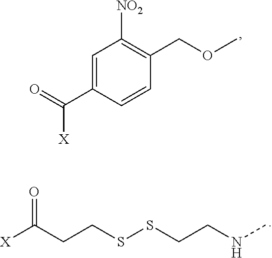

or covalent bond.

In some embodiments of the compound having the structure of formula (I), X is

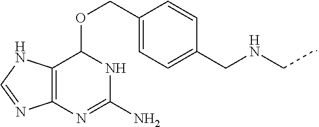

In some embodiments, X is

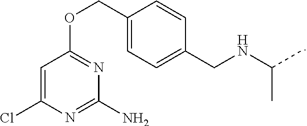

In some embodiments, X is

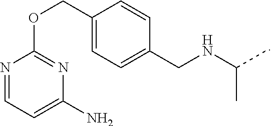

In some embodiments, X is

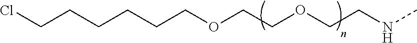

where n is 1-15.

In some embodiments, the compound has the following structure:

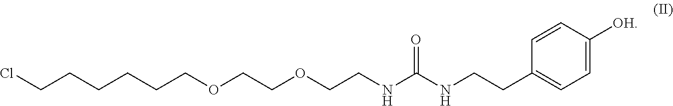 [SMILES: O=C(NCCOCCOCCCCCCCl)NCCc1ccc(O)cc1]

In some embodiments, the compound has the following structure:

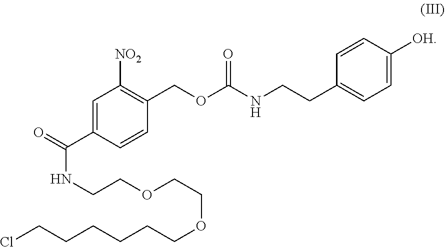 [SMILES: O=C(NCCc1ccc(O)cc1)OCc1ccc(C(=O)NCCOCCOCCCCCCCl)cc1[N+](=O)[O-]]

In some embodiments, the compound has the following structure:

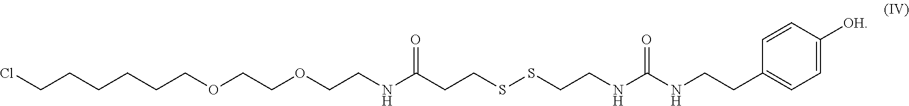 [SMILES: O=C(CCSSCCNC(=O)NCCc1ccc(O)cc1)NCCOCCOCCCCCCCl]

The presently-disclosed subject matter further includes a biotin-free method of labeling biomolecules in proximity to a target in a cell. In some embodiments, the method involves attaching an engineered labeling enzyme to the target. The method further involves contacting the cell with the compound as disclosed herein, which is a substrate of the engineered labeling enzyme and which includes a label moiety, wherein the biomolecules in proximity to the target are labeled. In some embodiments, the method also involves isolating the labeled biomolecules using a binding protein that binds to the labeled biomolecules.

Some embodiments of the method additionally include labeling the labeled biomolecules with a detectable label. In some embodiments, the detectable label is fluorescent, bioluminescent, or chemiluminescent. In some embodiments, the method also involves imaging luminescence emitted from the detectable label. In some embodiments, the method also involves identifying at least one labeled biomolecule.

In some embodiments of the method, the cell is an animal or a plant cell. In some embodiments, the cell is a live cell. In some embodiments, the cell is a fixed cell. In some embodiments, the cell is in an animal or plant. In some embodiments, the animal is living.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 presents the workflow of proximity labeling using chloroalkane (CLA)-phenol, an exemplary compound provided in accordance with the presently-disclosed subject matter.

FIGS. 2A-2C present data showing CLA-phenol labeling of neuronal lysosomal proteins. FIG. 2A includes images of primary neuron cultures and FIG. 2C includes zoomed-in images of these cultures. FIG. 2B includes a Western blot of primary neuron cultures.

FIGS. 3A-3D present data showing CLA-tyramide labeling in cilia (FIGS. 3A-3B) and mitochondria (FIGS. 3C-3D). FIGS. 3A and 3C are control conditions in which no ligand, or CLA-tyramide was used. FIGS. 3B and 3D are from experiments in which CLA-tyramide was applied to cells. In FIG. 3B, the image with the square border in the upper right-hand corner was a magnified image from the area with the smaller square border.

FIGS. 4A-4B present data showing CLA-tyramide (CLA-phenol) mediated proximity labeling in the mouse brain. FIG. 4A shows expression of APEX2, a peroxidase, in the mouse brain. FIG. 4B shows proximity labeling using CLA-tyramide.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds and methods for biotin-free proximity labeling.

The presently disclosed subject matter includes a compound having the following structure of formula (I):

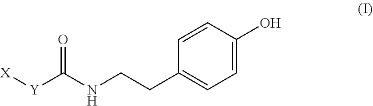

wherein X is a self-labeling protein ligand, and Y is

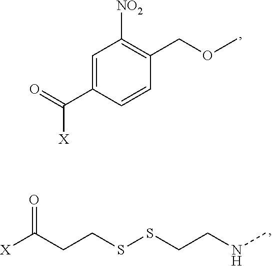

or covalent bond.

A “self-labeling protein” is a protein that can covalently attach to a specific chemical substrate, referred to as a ligand or self-labeling protein ligand. A “self-labeling protein ligand” is a chemical compound that specifically binds to a self-labeling protein, forming a stable covalent bond. These ligands can be linked to chemicals or functional groups. Together, the self-labeling protein and the self-labeling protein ligand provide a system for facilitating the specific attachment of a compound or functional group to a protein within a living cell or in vitro. The term “self-labeling” indicates that the protein is capable of catalyzing the attachment to the compound without the need for additional enzymes or co-factors.

A self-labeling protein/ligand system includes the self-labeling protein (SLP) (sometimes referred to in the art as a self-labeling protein tag) and the SLP ligand. The SLP and the SLP ligand form a specific bond. In this regard, when the SLP ligand is attached to a compound or functional group, the SLP forms a bond with the compound or functional group via the SLP ligand. This bond formation ensures a stable and irreversible attachment of the compound or functional group to the protein, allowing for various applications such as visualization, purification, and interaction studies.

Examples of SLPs and their SLP ligands will be known to those of ordinary skill in the art. One such example is HaloTag®, which is a modified bacterial enzyme that binds covalently to synthetic ligands containing a chloroalkane (CLA) moiety. Another example is SNAP-tag®, which is derived from the human DNA repair protein 06-alkylguanine-DNA alkyltransferase (AGT), and binds covalently to derivatives of its substrate 06-benzylguanine (BG). Another example is CLIP-tag®, which is similar to SNAP-tag®, but it binds to 02-benzylcytosine (BC) derivatives, allowing for orthogonal labeling in combination with SNAP-tag®. Another example is TMP-tag®, which is a self-labeling protein that binds to trimethoprim (TMP) derivatives. Another example is a tetracysteine tag, which is a peptide sequence that binds to biarsenical dyes like fluorescein arsenical helix binder (FLASH) and Resorufin Arsenical Helix binder (ReAsH), which are useful for fluorescent labeling. Another example is βLac-tag, which is a self-labeling protein derived from β-lactamase, which is often used with a ligand that is a β-lactam antibiotic, such as cephalosporin or penicillin derivatives. Another example is avidin-biotin, in which avidin (or streptavidin) can be provided as the self-labeling protein, which binds with high affinity and specificity to the ligand biotin, which is a vitamin (B7) that can be covalently attached to proteins, nucleic acids, or other compounds or functional groups.

In some embodiments of the compound having the structure of formula (I), X is

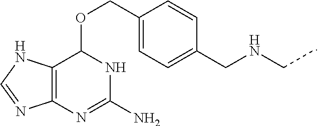

In some embodiments, X is

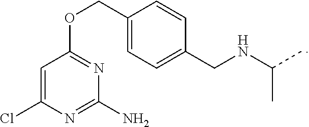

In some embodiments, X is

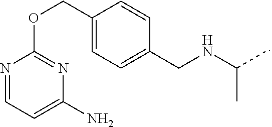

In some embodiments, X is

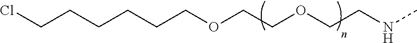

where n is 1, 2, 3, 4, 5, 6, 7, 8, or 9. In some embodiments, n can be 10-15. As will be appreciated by one of ordinary skill in the art, where n is larger, the resulting compound could become cell impermeable.

In some embodiments, the compound has the following structure:

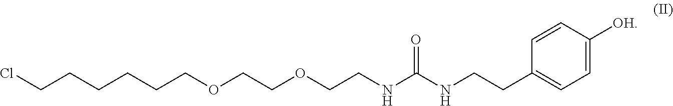 [SMILES: O=C(NCCOCCOCCCCCCCl)NCCc1ccc(O)cc1]

For some applications, it is preferable for the compound to be cell permeable. Some embodiments of the presently-disclosed compound are cell permeable. Some embodiments of the presently-disclosed compound can cross the blood-brain barrier (BBB). For some applications, it is preferable for the compound to be cell impermeable. For example, cell impermeable embodiments can be used for labeling biomolecules on the cell surface.

In some embodiments, the compound has the following structure:

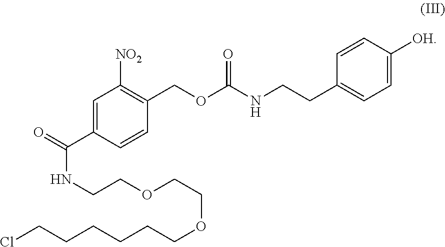 [SMILES: O=C(NCCc1ccc(O)cc1)OCc1ccc(C(=O)NCCOCCOCCCCCCCl)cc1[N+](=O)[O-]]

In some embodiments, the compound is cleaved into the following compounds when exposed to light:

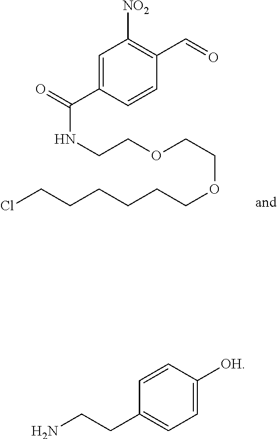 [SMILES: O=Cc1ccc(C(=O)NCCOCCOCCCCCCCl)cc1[N+](=O)[O-] ; NCCc1ccc(O)cc1]

In some embodiments, the compound has the following structure:

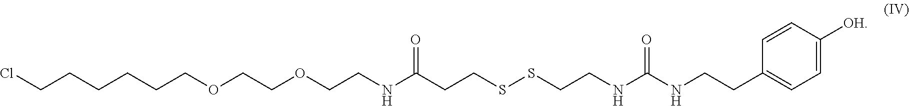 [SMILES: O=C(CCSSCCNC(=O)NCCc1ccc(O)cc1)NCCOCCOCCCCCCCl]

In some embodiments, the compound is thiol-cleavable.

The presently-disclosed subject matter further includes a biotin-free method of labeling biomolecules in proximity to a target in a cell. In some embodiments, the method involves attaching an engineered labeling enzyme to the target, and contacting the cell with a compound as disclosed hereinabove.

An “engineered labeling enzyme” is a modified enzyme designed to label molecules, such as proteins, in its vicinity. These enzymes catalyze reactions that attach a detectable marker to nearby proteins, allowing for the identification and study of protein interactions and localizations within cells. Because the present-disclosed method is biotin-free, for at least the reasons disclosed herein, biotin ligase (BirA) should not be used as the engineered labeling enzyme. There are various examples of engineered labeling enzymes that can be used in accordance with the presently-disclosed subject matter. One example is horseradish peroxidase (HRP), which catalyzes the oxidation of substrates like tetramethylbenzidine (TMB) in the presence of hydrogen peroxide, producing a colorimetric or chemiluminescent signal. Another example is ascorbate peroxidase (APEX), which is an enzyme that catalyzes the oxidation of ascorbate by hydrogen peroxide, producing a radical that can label nearby proteins with biotin-phenol. Additional examples include APEX2 peroxidase and dAPEX2 peroxidase. (Lam et al, 2015, Zhang et al, 2019).

As noted above, the biotin-free method of labeling biomolecules in proximity to a target in a cell involves attaching the engineered labeling enzyme to the target. In some embodiments, the target is a protein of interest. In some embodiments, the target is an organelle with or without a membrane. In some embodiments, the target is lysosome, nucleus, mitochondria, primary cilia, peroxisome, centrosome, ciliary rootlets, or a nucleolus.

Attaching the engineered labeling enzyme to the target can be achieved using methods known to those of ordinary skilling the art. For example, when the target is a protein-of-interest, genes encoding an enzyme/protein-of-interest fusion can be introduced into the cell(s) for expression, whereby the protein-of-interest will localize to its native environment within the cell. For another example, when the target is an organelle, the enzyme can be fused to a protein or a peptide sequence that preferentially delivers the enzyme to the organelle. In this regard, if the organelle was a lysosme, a lysosome-targeted enzyme could be provided by fusing Lysosome-Associated Membrane Protein 1 (Lamp1) to the enzyme. Other targeting sequences exist for other organelles. See, e.g., Lam et al. (2015) and Rhee et al. (2013).

As noted above, the biotin-free method of labeling biomolecules in proximity to a target in a cell further involves contacting the cell with a compound as disclosed hereinabove, which includes a phenol and a self-labeling protein ligand. The compound is a substrate of the engineered labeling enzyme (e.g., at least because it includes a phenol) and includes a label moiety (e.g., at least because it includes a self-labeling protein ligand), wherein the biomolecules in proximity to the target are labeled.

In some embodiments, the method also involves isolating the labeled biomolecules using a binding protein that binds to the labeled biomolecules. The method can involve, in some embodiments, isolating the labeled biomolecules using a binding protein that binds to the labeled biomolecules. In some embodiments, the binding protein can be immobilized on a solid support.

For example, the biomolecules can be labeled with the self-labeling protein ligand, and the self-labeling protein can be used to bind and isolate the labeled biomolecules. In this regard, in some embodiments, the binding protein is a self-labeling protein and the label moiety includes a self-labeling protein ligand, as disclosed herein. In this regard, in some embodiments, the self-labeling protein ligand includes a chloroalkane (CLA) moiety, and the self-labeling protein has a site that reacts specifically with the CLA moiety. In this regard, the self-labeling protein can be HaloTag®. In some embodiments, the self-labeling protein ligand includes a benzylguanine moiety, and the self-labeling protein has a site that reacts specifically with the benzylguanine moiety. In this regard, the self-labeling protein can be SNAP-Tag®. In some embodiments, the self-labeling protein ligand includes a benzylcytosine moiety, and the self-labeling protein has a site that reacts specifically with the benzylcytosine moiety. In this regard, the self-labeling protein can be CLIP-Tag®.

In some embodiments, the method further involves contacting the cell with hydrogen peroxide. In some embodiments, the step of contacting the cell with the compound as disclosed herein (which is a substrate of the engineered labeling enzyme and which includes a label moiety) is performed in 10 minutes or less. In some embodiments, the method also includes contacting the cell with an antioxidant buffer.

Some embodiments of the method additional include labeling the labeled biomolecules with a detectable label. For example, a biomolecule that has been labeled with a self-labeling protein tag ligand (e.g., CLA) can be further labeled with a detectable label. In some embodiments, a label moiety can comprise a self-labeling protein tag ligand and a detectable label. In some embodiments, a binding protein and further comprise a detectable label.

In some embodiments, the detectable label is fluorescent, bioluminescent, or chemiluminescent. In some embodiments, the method also involves imaging luminescence emitted from the detectable label. In some embodiments, the method also involves identifying at least one labeled biomolecule. In some embodiments, the identifying comprises performing mass spectrometry (MS), liquid chromatography-mass spectrometry (LC/MS), an enzyme-linked immunosorbent assay (ELISA), a Western blot, immunostaining, high-performance liquid chromatography (HPLC), protein sequencing, or peptide mass fingerprinting.

In some embodiments of the method, the cell is an animal or a plant cell. In some embodiments, the cell is a live cell. In some embodiments, the cell is a fixed cell. In some embodiments, the cell is in an animal or plant. In some embodiments, the animal is living.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

An exemplary system provided in accordance with the presently-disclosed subject matter was tested to establish efficacy. In this Example, an exemplary system was tested, which involved use of a compound including a moiety that is a ligand of the self-labeling protein HaloTag. HaloTag is bacterially-derived and, as distinguished from biotin, its chloroalkane (CLA) ligands are not known to be present in eukaryotic cells.

The exemplary system studied in this Example makes use of a unique substrate for proximity labeling, which is a CLA-phenol compound, which in some cases is referred to herein as a CLA-tyramide compound. As will be appreciated, the chemical structure of tyramide includes a phenol moiety. To establish efficacy, the exemplary system was used to detect proteins that are enriched in neuronal lysosomes with lysosome-targeted APEX2 (Lamp1-APEX2).

Synthesis of CLA-phenol was performed as follows. N,N′-Disuccinimidyl carbonate (374 mg, 1.46 mmol, 1.5 equiv), tyramine (200 mg, 1.46 mmol, 1.5 equiv), and N, N-diisopropylethylamine (DIEA; 540 μL, 2.91 mmol, 3 equiv) were added to anhydrous DMF (5 mL). The reaction mixture was stirred for 60 min at ambient temperature, after which HaloTag (O₂)—NH₂·HCl (250 mg, 0.96 mmol, 1 equiv) and DIEA (540 μL, 2.91 mmol, 3 equiv) were added. The reaction mixture was stirred for 16 hours at ambient temperature. The solvent was removed under reduced pressure and the crude was purified by preparative HPLC using a 5-95% CH₃CN/H₂O linear gradient with a constant 0.1% v/v TFA. Product-containing fractions were combined and lyophilized to obtain a light-yellow oil (207.5 mg, 56%). ¹H NMR (400 MHz, CD₃CN) δ 7.02-6.99 (m, 2H), 6.75-6.71 (m, 2H), 3.55 (t, J=6.7 Hz, 2H), 3.53-3.47 (m, 4H), 3.43 (t, J=5.5 Hz, 2H), 3.39 (t, J=6.6 Hz, 2H), 3.27 (dd, J=7.9, 6.6 Hz, 2H), 3.22 (t, J=5.5 Hz, 2H), 2.63 (t, J=7.2 Hz, 2H), 1.76-1.69 (m, 2H), 1.52 (p, J=6.8 Hz, 2H), 1.44-1.28 (m, 4H). ¹³C NMR (101 MHZ, CD₃CN) δ 159.99 (C), 156.54 (C), 131.29 (CH), 130.72 (C), 116.19 (CH), 71.69 (CH₂), 71.14 (CH₂), 70.97 (CH₂), 70.71 (CH₂), 46.21 (CH₂), 42.72 (CH₂), 40.90 (CH₂), 36.23 (CH₂), 33.33 (CH₂), 30.25 (CH₂), 27.39 (CH₂), 26.14 (CH₂). HRMS (ESI) calculated for C19H32CIN23O4 [M+H]⁺=387.2045, found 387.2043.

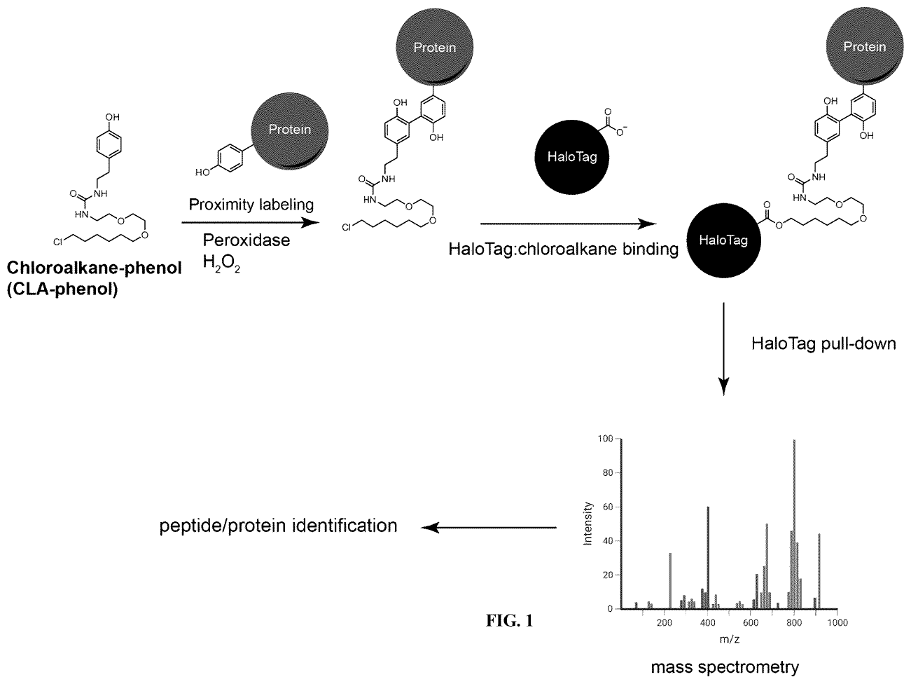

With reference to FIG. 1, a compound is provided in accordance with the presently-disclosed subject matter. In this example, the compound is of formula (I), including a phenol, and the label moiety that is the self-labeling protein ligand chloroalkane (CLA), which is a HaloTag® ligand. Accordingly, in FIG. 1, the compound is labeled CLA-phenol.

In the example depicted in FIG. 1, the engineered labeling enzyme that is used can be an ascorbate peroxidase, such as APEX2. In the presence of APEX2 and hydrogen peroxide, the CLA-phenol compound readily attaches to nearby proteins. A binding protein can be used to bind the labeled protein molecules. In the example depicted in FIG. 1, the binding protein is the self-labeling protein HaloTag®, where the protein molecules have been labeled with CLA, a ligand of HaloTag®.

As further depicted in FIG. 1, the binding protein (e.g., HaloTag®) can be used to isolate the labeled molecules (e.g., CLA-protein). As will be appreciated by one of ordinary skill in the art, in some cases it can be useful to immobilize the binding protein on a solid support. As depicted in FIG. 1, HaloTag® pull down of the labeled molecule (e.g., protein) can occur so that the molecule can be analyzed and/or identified (e.g., using mass spectrometry analyses).

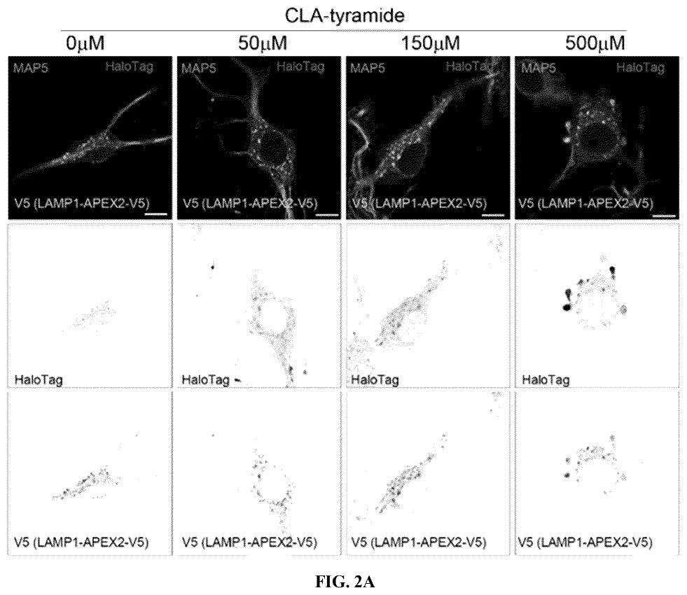

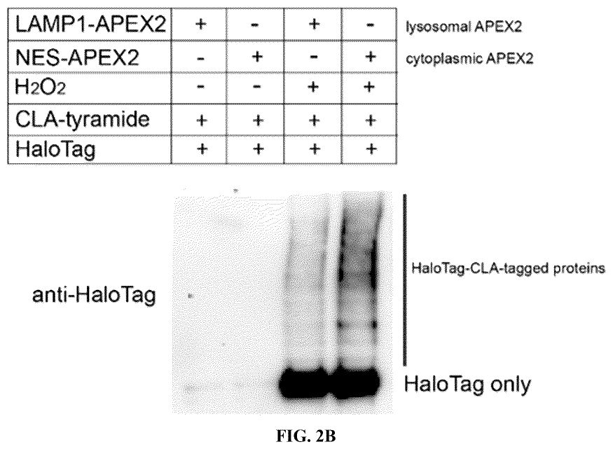

With reference to FIG. 2A, cultured primary neurons expressing LAMP1-APEX2-V5 were incubated with 0 μM, 50 μM, 150 μM, or 500 μM CLA-tyramide, respectively. Neurons were fixed, permeabilized, and incubated with HaloTag protein. Cells were then stained with antibodies against HaloTag, V5, and MAP5 to label CLA-tagged proteins, LAMP1-APEX2-V5 (lysosome APEX2), and neurons, respectively.

FIG. 2B includes a Western blot of cultured primary neurons expressing LAMP1-APEX2-V5 or NES-APEX2-V5. In groups with H₂O₂ stimulation (right two lanes), antibody against HaloTag showed HaloTag only and various CLA-tagged proteins (smear signals above HaloTag).

FIG. 2C includes zoomed-in images from cultures shown in FIG. 2A. Line scans show the relative intensity of CLA-tagged proteins and LAMP1-APEX2-V5. Good colocalization was detected in 150 μM CLA-tyramide incubated (right) but not in 0 μM CLA-tyramide incubated samples (controls, left).

Using CLA-phenol labeling, the neuronal lysosomal proteins presented in Table 1 were identified.

TABLE 1
Proteins enriched in lysosomes detected by mass spectrometry. Each
field denotes the UniProt ID, and the name of the protein separated by |.

| | |
|---|---|
| P09951\|SYN1_RAT | Q00715\|H2B1_RAT |
| P62815\|VATB2_RAT | P11505\|AT2B1_RAT |
| P07825\|SYPH_RAT | P05426\|RL7_RAT |
| P46462\|TERA_RAT | P47942\|DPYL2_RAT |
| Q5U206\|CALL3_RAT | P97710\|SHPS1_RAT |
| PODP31\|CALM3_RAT | Q9WVC0\|SEPT7_RAT |
| PODP30\|CALM2_RAT | F1LX07\|S2512_RAT |
| PODP29\|CALM1_RAT | Q62950\|DPYL1_RAT |
| P05065\|ALDOA_RAT | P62804\|H4_RAT |
| P37805\|TAGL3_RAT | Q4QRB4\|TBB3_RAT |
| P34926\|MAP1A_RAT | P63259\|ACTG_RAT |
| Q63429\|UBC_RAT | P60711\|ACTB_RAT |
| POCG51\|UBB_RAT | P15865\|H14_RAT |
| P62986\|RL40_RAT | P19332\|TAU_RAT |
| P62982\|RS27A_RAT | Q63537\|SYN2_RAT |
| P45592\|COF1_RAT | Q63716\|PRDX1_RAT |
| P63018\|HSP7C_RAT | P10719\|ATPB_RAT |
| P47819\|GFAP_RAT | Q9JK11\|RTN4_RAT |
| Q6AYH5\|DCTN2_RAT | P48500\|TPIS_RAT |

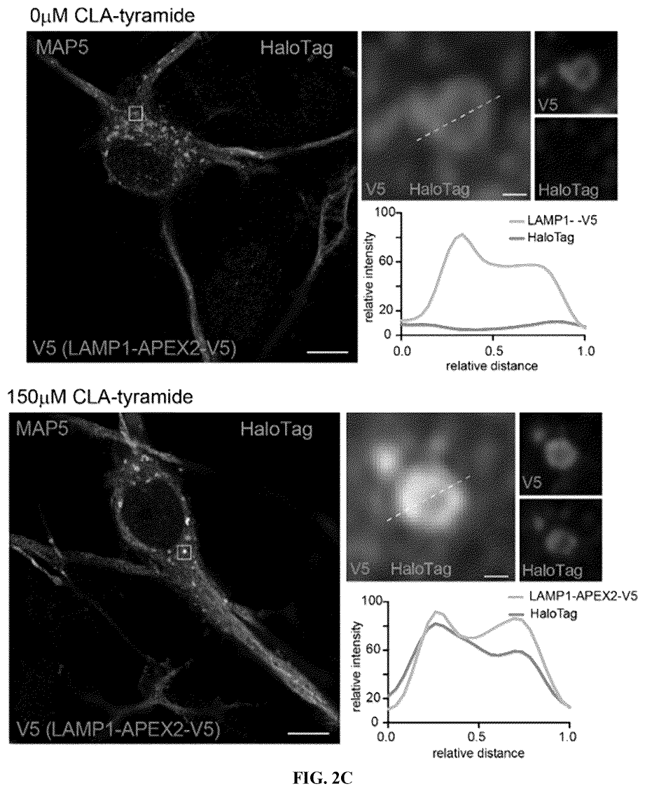

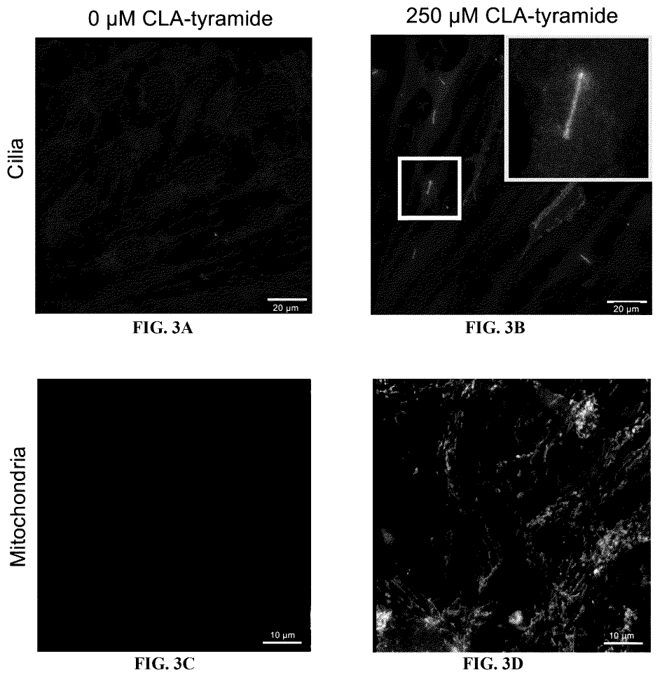

CLA-tyramide labeling was conducted in cilia and mitochondria. FIGS. 3A and 3B show CLA-tyramide labeling in cilia. FIGS. 3C and 3D show CLA-tyramide labeling in mitochondria. Control conditions in which no ligand or CLA-tyramide was used are shown in FIGS. 3A and 3C. FIGS. 3B and 3D are from experiments in which 250 μM CLA-tyramide was applied to cells. Cells were fixed, permeabilized, and incubated with EGFP-HaloTag protein. These examples showed that the method can be applied to different subcellular compartments.

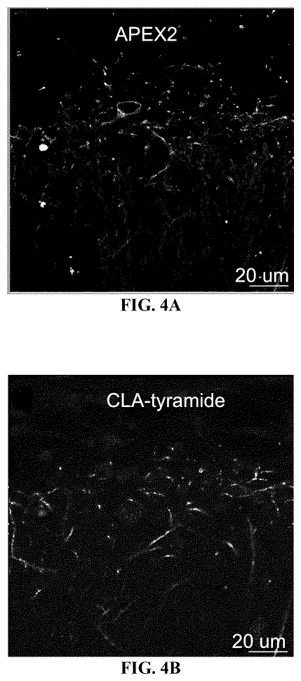

CLA-tyramide (CLA-phenol) mediated proximity labeling was conducted in mouse brain. FIG. 4A shows expression of APEX2, a peroxidase, in the mouse brain. The mouse brain samples were fixed, permeabilized, and incubated with HaloTag protein. The samples were then stained with antibodies against HaloTag and V5 (on APEX2) to label CLA-tagged proteins and APEX2, respectively. FIG. 4B shows proximity labeling using CLA-tyramide. They showed good co-localization of most structures.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Lam S S, Martell J D, Kamer K J, Deerinck T J, Ellisman M H, Mootha V K, Ting A Y. Directed evolution of APEX2 for electron microscopy and proximity labeling. Nat Methods. 2015 January; 12 (1): 51-4. doi: 10.1038/nmeth.3179. Epub 2014 Nov. 24. PMID: 25419960; PMCID: PMC4296904.
2. Rhee H W, Zou P, Udeshi N D, Martell J D, Mootha V K, Carr S A, Ting A Y. Proteomic mapping of mitochondria in living cells via spatially restricted enzymatic tagging. Science. 2013 Mar. 15; 339 (6125): 1328-1331. doi: 10.1126/science.1230593. Epub 2013 Jan. 31. PMID: 23371551; PMCID: PMC3916822,
3. Zhang Q, Lee W A, Paul D L, Ginty D D. Multiplexed peroxidase-based electron microscopy labeling enables simultaneous visualization of multiple cell types. Nat Neurosci. 2019 May; 22 (5): 828-839. doi: 10.1038/s41593-019-0358-7. Epub 2019 Mar. 18. PMID: 30886406; PMCID: PMC6555422.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound of the formula: 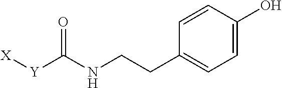
wherein
X is a self-labeling protein ligand, and
Y is 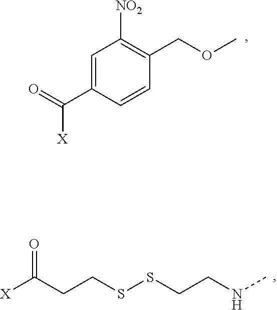
or covalent bond.

2. The compound of claim 1, wherein
X is 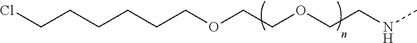
where n is 1-9,

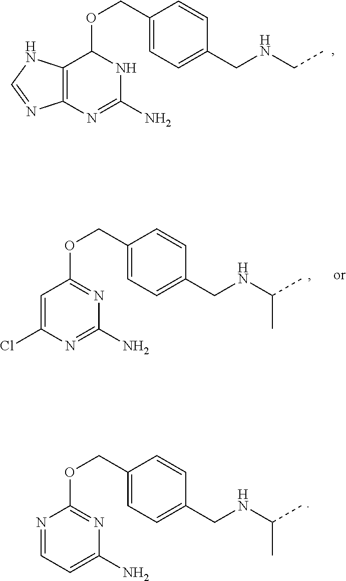

3. The compound of claim 1, according to the following formula

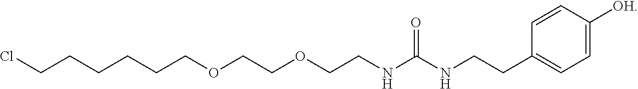 [SMILES: O=C(NCCOCCOCCCCCCCl)NCCc1ccc(O)cc1]

4. The compound of claim 3, which is cell permeable and blood-brain barrier (BBB) permeable.

5. The compound of claim 1, according to the following formula 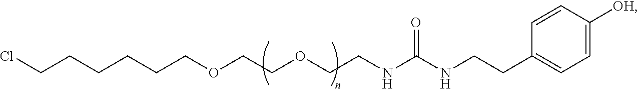
wherein n is 1-9.

6. The compound of claim 1, according to the following formula

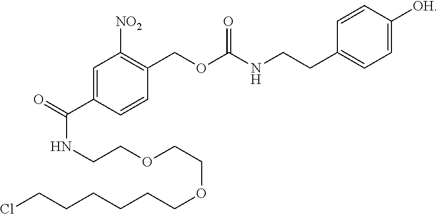 [SMILES: O=C(NCCc1ccc(O)cc1)OCc1ccc(C(=O)NCCOCCOCCCCCCCl)cc1[N+](=O)[O-]]

7. The compound of claim 1, according to the following formula

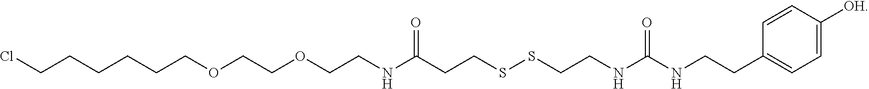 [SMILES: O=C(CCSSCCNC(=O)NCCc1ccc(O)cc1)NCCOCCOCCCCCCCl]

8. A biotin-free method of labeling biomolecules in proximity to a target in a cell, comprising:
(a) attaching an engineered labeling enzyme to the target; and
(b) contacting the cell with the compound of claim 1, which is a substrate of the engineered labeling enzyme and which comprises a label moiety, wherein the biomolecules in proximity to the target are labeled.

9. The method of claim 8, further comprising isolating the labeled biomolecules using a binding protein that binds to the labeled biomolecules.

10. The method of claim 9, wherein the binding protein is immobilized on a solid support.

11. The method of claim 9, wherein the binding protein is a self-labeling protein and the label moiety comprises a self-labeling protein ligand.

12. The method of claim 11, wherein the self-labeling protein ligand comprises a chloroalkane (CLA) moiety, and the self-labeling protein has a site that reacts specifically with the CLA moiety.

13. The method of claim 11, wherein the self-labeling protein ligand comprises a benzylguanine moiety, and the self-labeling protein has a site that reacts specifically with the benzylguanine moiety.

14. The method of claim 10, wherein the engineered labeling enzyme is an ascorbate peroxidase or horseradish peroxidase.

15. The method of claim 14, wherein the ascorbate peroxidase is APEX peroxidase or APEX2 peroxidase.

16. The method of claim 15, and further comprising contacting the cell with hydrogen peroxide.

17. The method of claim 16, wherein step (b) is performed in 10 minutes or less.

18. The method of claim 17, and further comprising contacting the cell with an antioxidant buffer.

19. The method of claim 10, further comprising additionally labeling the labeled biomolecules with a detectable label.

20. The method of claim 19, further comprising imaging signal emitted from the detectable label.
